SEQUENCE LISTING (5 sequences)

SEQ ID NO: 1 - protein, 311 aa, Homo sapiens
MLTKPLQGPPAPPGTPTPPPGGKDREAFEAEYRLGPLLGKGGFGTVFAGHRLTDRLQVAIKVIPRNRVLGWSPLSDSVTCPLEVALLWKVGAGGGHPGVIRLLDWFETQEGFMLVLERPLPAQDLFDYITEKGPLGEGPSRCFFGQVVAAIQHCHSRGVVHRDIKDENILIDLRRGCAKLIDFGSGALLHDEPYTDFDGTRVYSPPEWISRHQYHALPATVWSLGILLYDMVCGDIPFERDQEILEAELHFPAHVSPDCCALIRRCLAPKPSSRPSLEEILLDPWMQTPAEDVPLNPSKGGPAPLAWSLLP

SEQ ID NO: 2 - DNA, 23 bp, Homo sapiens
gactccaggtatccgtcatgagg

SEQ ID NO: 3 - DNA, 23 bp, Artificial
  Synthetic splice switching antisense oligonucleotide for PIM2
cctcatgacggatacctggagtc

SEQ ID NO: 4 - protein, 452 aa, Artificial
  Synthetic Daratumumab heavy chain
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, Artificial
  Synthetic Daratumumab light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0368605 A1
(43) Pub. Date: Nov. 7, 2024

(54) USE OF SPLICE SWITCHING OLIGONUCLEOTIDES FOR EXON SKIPPING-MEDIATED KNOCKDOWN OF PIM2

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); ETABLISSEMENT FRANÇAIS DU SANG (EFS), La Plaine Saint Denis (FR); UNIVERSITÉ DE LIMOGES, Limoges (FR); UNIVERSITÉ DE MONTPELLIER, Montpellier (FR); UNIVERSITE DE RENNES, Rennes (FR)

(72) Inventors: Thierry FEST, Rennes Cedex (FR); Laurent DELPY, Limoges (FR); Marion HAAS, Rennes Cedex (FR); Anne MARCHALOT, Huddinge (SE); Gersende LACOMBE, Rennes Cedex (FR); Jérôme MOREAUX, Montpellier (FR)

(21) Appl. No.: 18/285,693

(22) PCT Filed: Apr. 15, 2022
(86) PCT No.: PCT/EP2022/060187

§ 371 Date: Oct. 5, 2023

(30) Foreign Application Priority Data
Apr. 19, 2021 (EP) 21305513.0

(51) Int. Cl. C12N 15/113

(52) U.S. Cl. CPC C12N 15/1137; C12N 2310/11; C12N 2320/33

(57) ABSTRACT

PIM2 kinase deregulation has been reported in several cancers. In particular, PIM2 is considered in multiple myeloma as part of the oncogenic process and several PIM kinase inhibitors have been developed showing encouraging results in preclinical studies and clinical trials. Now the inventors have developed an antisense RNA strategy based on a splice-switching oligonucleotide (SSO) so as to induce efficient knockdown of PIM2 expression. This SSO-mediated knockdown is a powerful approach to for cancer treatments. Accordingly, the present invention relates to the use of splice switching oligonucleotides for exon skipping-mediated knockdown of PIM2.

### FIELD OF THE INVENTION

The present invention is in the field of medicine, in particular oncology.

### BACKGROUND OF THE INVENTION

During immune responses, antibody secretion is produced by a rare population of terminal differentiated B cells known as plasma cells (PCs). Along the PC route, B cells first pass through an immature and proliferative plasmablast stage. These cells emerge after vast modifications in the morphology, epigenome-sustained expression profile, and lifespan compared with their B cell predecessors (Fairfax et al., 2008; Willis and Nutt, 2019). This cell metamorphosis follows a tightly regulated process that can be hijacked by oncogenic alterations driving malignant transformation. Thus, the malignancy may take place either in the molecular plasmacytic network as described in post-GC lymphomas or in features related to PC biology for multiple myeloma (MM) (Pawlyn and Morgan, 2017; Shaffer et al., 2012). Despite a rich literature regarding normal B cell differentiation, insights from B cell malignancies are revealing holes in our understanding of normal B cell function (Shaffer et al., 2012). For instance, regarding how newly generated plasmablasts (PBs) metabolically acquire their secretory capacity and mount ER stress responses is only partially understood (Lam et al., 2018). One difficulty resides in our capacity to identify specific factors and their spatio-temporal involvement in molecular modifications sustaining last steps of the B cell metamorphosis.

Recently, we revealed that the emergence of human PBs is associated with large-scale methylome modifications with local acquisition of genome active marks at PC identity genes during a committal step when, in vitro, one dividing B cell differentiate into two PBs (Caron et al., 2015). These committed B cells, called hereafter pre-plasmablasts (prePBs), belong to a highly proliferative subset of activated B cells characterized by the downregulation of the CD23 surface marker due to the silencing of the IL4/STAT6 pathway (Pignarre et al., 2021). Using this feature, a comparative RNA-seq analysis identified in prePBs a striking increase of expression of the gene encoding the serine/threonine protein kinase PIM2 which belongs to the PIM family kinases described previously as associated with B cell survival and proliferation (Mondello et al., 2014). These constitutively active kinases were originally described as proto-oncogenes activated by retroviral insertion resulting in aberrant overexpression (Theo Cuypers et al., 1984). Molecular evidence has emerged linking PIMs to specific phosphorylation events associated with cell cycle checkpoints and expression of anti-apoptotic proteins (Mondello et al., 2014). Despite these associations, animals lacking Pim kinases are viable, fertile with a minimal immune phenotype (Mikkers et al., 2004). However, (publicly accessible Penn dissertation, http://repository.upenn.edu/edissertations/165), John Treml showed that Pim1−/− Pim2−/− animals have little serum immunoglobulin, lack T-independent responses and reduce largely T-dependent immunogen responses while maintaining germinal center (GC) formation (Treml, 2010).

Deregulated PIM kinases have been reported in several cancers but unlike PIM1, PIM2 is rarely mutated. However, PIM2 is considered in multiple myeloma as part of the oncogenic process and several PIM kinase inhibitors have been developed showing encouraging results in preclinical studies and clinical trials (Gomez-Abad et al., 2011; Lu et al., 2013; Raab et al., 2019). Surprisingly, PIM2 has never been analyzed in the context of normal B cells differentiation into PBs and neither in the biology of mature PCs.

The regulation of cell survival has a well-documented role in the maintenance of homeostasis, the damage/stress response, and is misregulated in various disease states. Mouse pro-B cells analysis revealed that growth factor-induced transcription is dependent of endogenous levels of Pim2 which promotes cell survival. Indeed, cell size and mitochondrial potential are directly dependent to Pim2 and its ability to phosphorylated in a rapamycin-resistant manner the translational regulator 4E-BP1 and the BH3 protein Bad (Fox, 2003). The dynamic regulation of the constitutive catalytically-active PIM2 kinase by growth factor-dependent transcription raises questions, justifying further investigations in the context of PC differentiation including at the transcriptional level and the recruitment of cell-specific genomic regions called enhancers which are bound by transcription factors controlling the differentiation process. Indeed, the final commitment of B cells is associated with the accumulation of 5-hydroxymethylcytosine (5hmC) at PC identity genes, which sustains transcriptional modifications driving PB emergence (Caron et al., 2015).

### SUMMARY OF THE INVENTION

The present invention is defined by the claims. In particular, the present invention relates to the use of splice switching oligonucleotides for exon skipping-mediated knockdown of PIM2.

### DETAILED DESCRIPTION OF THE INVENTION

### Main Definitions

As used herein, the term “subject” refers to any mammals, such as a rodent, a feline, a canine, and a primate.

As used herein, the term “malignant cell” has its general meaning in the art and refers to a neoplastic or transformed cell. Typically, a malignant cell exhibits one or more characteristics or hallmarks of cancer. Such hallmarks of cancer include self-sufficiency in growth signals, insensitivity to growth-inhibitory (antigrowth) signals, evasion of programmed cell death (apoptosis), limitless replicative potential, sustained angiogenesis, and tissue invasion and metastasis.

As used herein, the term “B cell” has its general meaning in the art and is used herein to mean an immune cell that develops in the bone marrow and is highly specialized for making immunoglobulins and antibodies. A B cell is a lymphocyte which is derived from bone marrow and provides humoral immunity. A B cell recognizes antigen molecules in solution and matures into a plasma cell. Thus, when the term “B cell” is used herein it is intended to encompass cells developed from B cells such as plasmablasts and plasma cells.

As used herein, the term “plasma cell” has its general meaning in the art and is intended to mean a cell that develops from a B lymphocyte in reaction to a specific antigen. Plasma cells are found in bone marrow and blood and secrete large amounts of antibodies. Plasma cells differentiate from B cells upon stimulation by CD4+ lymphocytes. A plasma cell is a type of white blood cell that produces antibodies and is derived from an antigen-specific B cell.

As used herein, the terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “PIM2” has its general meaning in the art and refers to the serine/threonine-protein kinase pim-2. An exemplary amino acid sequence for PIM2 is shown as SEQ ID NO:1.

>sp|Q9P1W9|PIM2_HUMAN Serine/threonine-protein
kinase pim-2 OS = Homo sapiens OX = 9606
GN = PIM2 PE = 1 SV = 1
SEQ ID NO: 1
MLTKPLQGPPAPPGTPTPPPGGKDREAFEAEYRLGPLLGKGGFGTVF
AGHRLTDRLQVAIKVIPRNRVLGWSPLSDSVTCPLEVALLWKVGAGG
GHPGVIRLLDWFETQEGFMLVLERPLPAQDLFDYITEKGPLGEGPSR
CFFGQVVAAIQHCHSRGVVHRDIKDENILIDLRRGCAKLIDFGSGAL
LHDEPYTDFDGTRVYSPPEWISRHQYHALPATVWSLGILLYDMVCGD
IPFERDQEILEAELHFPAHVSPDCCALIRRCLAPKPSSRPSLEEILL
DPWMQTPAEDVPLNPSKGGPAPLAWSLLP

As used herein, the expression “reducing the expression of PIM2” means a measurable decrease in the number of said PIM2 in a cell (e.g. a B cell). The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of said PIM2 to an amount below detectable limit. Methods for quantifying expression of PIM2 are well known in the art and typically include those described in the EXAMPLE.

As used herein, the term “pre-mRNA”, “precursor mRNA” or “primary RNA transcript” refers to a strand of messenger ribonucleic acid (mRNA), synthesized from a DNA template in the nucleus of a cell by transcription, prior to processing events such as splicing. Generally, eukaryotic pre-mRNA exists only briefly before it is fully processed into mature mRNA. Pre-mRNA includes two different types of segments, exons and introns. Most of exons encode protein, while introns are usually excised before translation by a process known as “splicing”.

As used herein, the term “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA.

As used herein, the term “intron” refers to a nucleic acid region (within a gene) that is not translated into a protein. An intron is a non-coding section that is transcribed into a precursor mRNA (pre-mRNA), and subsequently removed by splicing during formation of the mature RNA.

As used herein, the term “splice site” in the context of a pre-mRNA molecule, refers to the short-conserved sequence at the 5′ end (donor site) or 3′ end (acceptor site) of an intron to which a spliceosome binds and catalyzes the splicing of the intron from the pre-mRNA.

As used herein, the term “exon skipping” refers generally to the process by which an entire exon, or a portion thereof, is removed from a given pre-processed RNA, and is thereby excluded from being present in the mature RNA. According to the present invention the exon deletion leads to a reading frame shift in the shortened transcribed mRNA that would lead to the generation of truncated non-functional protein or nonsense-mediated decay (NMD) degradation.

As used herein, the term “antisense oligonucleotide” or “ASO” refers to a single strand of DNA, RNA, or modified nucleic acids that is complementary to a chosen sequence. Antisense RNA can be used to prevent protein translation of certain mRNA strands by binding to them. Antisense DNA can be used to target a specific, complementary (coding or non-coding) RNA. Such an antisense oligomer can be designed to block or inhibit translation of mRNA or to inhibit natural pre-mRNA splice processing, and may be said to be “directed to” or “targeted against” a target sequence with which it hybridizes. According to the present invention, the target sequence is a splice site of a pre-processed mRNA. In said embodiments, the ASO is named as a “splice switching antisense oligonucleotide” or “SSO”. For instance, the target sequence for a splice site may include an mRNA sequence having its 5′ end 1 to about 25 base pairs downstream of a normal splice acceptor junction in a preprocessed mRNA. A preferred target sequence is any region of a precursor mRNA that includes a splice site or is contained entirely within an exon coding sequence or spans a splice acceptor or donor site or exon/intron regulatory sequences (ESE, ISE).

As used herein, the term “complementary” as used herein includes “fully complementary” and “substantially complementary”, meaning there will usually be a degree of complementarity between the oligonucleotide and its corresponding target sequence of more than 80%, preferably more than 85%, still more preferably more than 90%, most preferably more than 95%. For example, for an oligonucleotide of 20 nucleotides in length with one mismatch between its sequence and its target sequence, the degree of complementarity is 95%.

As used herein, the term “isolated” means material that is substantially or essentially free from components that normally accompany it in its native state. For example, an “isolated polynucleotide,” as used herein, may refer to a polynucleotide that has been purified or removed from the sequences that flank it in a naturally-occurring state, e.g., a DNA fragment that has been removed from the sequences that are normally adjacent to the fragment.

As used herein, the term “stabilized SSO” refers to a SSO that is relatively resistant to in vivo degradation (e.g., via an exo- or endo-nuclease).

As used herein, the term “B-cell malignancy” includes any type of leukemia or lymphoma of B cells.

As used herein, the term “B cell lymphoma” refers to a cancer that arises in cells of the lymphatic system from B cells.

As used herein, the term “multiple myeloma” as used herein means a disseminated malignant neoplasm of plasma cells which is characterized by multiple bone marrow tumor foci and secretion of an M component (a monoclonal immunoglobulin fragment), associated with widespread osteolytic lesions resulting in bone pain, pathologic fractures, hypercalcaemia and normochromic normocytic anaemia.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” is intended for a minimal amount of the active agent (i.e the SSO of the present invention) which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder.

### Methods of the Present Invention

An object of the present invention relates to a method of reducing the expression of PIM2 in a subject in need thereof comprising administering to the subject an effective amount of at least one splice switching antisense oligonucleotide targeting a splice site of one exon, or a splicing regulatory sequence in the pre-mRNA molecule encoding for PIM2 to alter splicing by blocking the recognition of said splice site by splicing machinery and thus inducing the exon skipping.

In some embodiments, the method of the present invention is particular suitable for reducing the expression of PIM2 in B cells, in particular in plasmablasts and/or plasma cells.

In some embodiments, the method of the present invention is particular suitable for reducing the expression of PIM2 in malignant cells. In some embodiments, the method of the present invention is particular suitable for reducing the expression of PIM2 in malignant B cells and/or malignant plasma cells.

According to the present invention the splice switching oligonucleotide (SSO) mediates the exon-skipping for a pre-mRNA having at least 3 exons with a targeted internal one having a number of nucleotides not divisible by 3 for inducing a reading frameshift. In addition, the exon skipping must provoke the appearance of a premature termination codon (PTC) to shorten drastically the open reading frame and/or support nonsense-mediated mRNA decay (NMD) degradation.

In some embodiments, the splice switching antisense oligonucleotide of the present invention is an antisense RNA.

In some embodiments, the splice switching antisense oligonucleotide of the present invention is an antisense DNA.

The length of the splice switching antisense oligonucleotide may vary so long as it is capable of binding selectively to the intended location within the pre-mRNA molecule. The length of such sequences can be determined in accordance with selection procedures described herein. Generally, the antisense molecule will be from about 10 nucleotides in length up to about 50 nucleotides in length. It will be appreciated however that any length of nucleotides within this range may be used in the method. Preferably, the length of the antisense molecule is between 10-30 nucleotides in length. In some embodiments, the splice switching antisense oligonucleotide of the present invention has a sufficient length. As used herein, “sufficient length” refers to an antisense oligonucleotide that is complementary to at least 8, more typically 8-30, contiguous nucleobases in the target pre-mRNA. In some embodiments, an antisense of sufficient length includes at least 8, 9, 10, 11, 12, 13, 14, 15, 17, 20 or more contiguous nucleobases in the target pre-mRNA. In some embodiments an antisense of sufficient length includes at least 16, 17, 18, 19, 20, 21, 22, 23, 24, or 25 contiguous nucleobases in the target pre-mRNA.

In some embodiments, the splice switching antisense oligonucleotide of the present invention targets the PIM2 exon 2 donor splice sites. In some embodiments, the splice switching antisense oligonucleotide of the present invention is complementary to the nucleic acid sequence as shown in SEQ ID NO:2. In some embodiments, the splice switching antisense oligonucleotide of the present invention converts the PIM2 protein translation into an inactive peptide lacking all active domains compared to full-length PIM2 isoform. In some embodiments, the splice switching antisense oligonucleotide of the present invention targets t the PIM2 exon 2 donor splice site and comprises the sequence as set forth in SEQ ID NO:3.

target sequence for PIM2
SEQ ID NO: 2
5′-GACTCCAGGTATCCGTCATGAGG-3′
splice switching antisense oligonucleotide for
PIM2
SEQ ID NO: 3
5′-CCTCATGACGGATACCTGGAGTC-3′

In some embodiments, the splice switching antisense oligonucleotide of the present invention is stabilized. Stabilization can be a function of length or secondary structure. Alternatively, SSO stabilization can be accomplished via phosphate backbone modifications. Preferred stabilized SSOs of the present invention have a modified backbone, e.g., have phosphorothioate linkages to provide maximal activity and protect the SSO from degradation by intracellular exo- and endo-nucleases. Other possible stabilizing modifications include phosphodiester modifications, combinations of phosphodiester and phosphorothioate modifications, methylphosphonate, methylphosphorothioate, phosphorodithioate, p-ethoxy, and combinations thereof. Chemically stabilized, modified versions of the SSO's also include “Morpholinos” (phosphorodiamidate morpholino oligomers, PMOs), 2′-O-Met oligomers, 2′Methoxy-ethyl oligomers, 2′-Fluoro (2′-F) oligomers, tricyclo (tc)-DNAs, U7 short nuclear (sn) RNAs, tricyclo-DNA-oligoantisense molecules (U.S. Provisional Patent Application Ser. No. 61/212,384 For: Tricyclo-DNA Antisense Oligonucleotides, Compositions and Methods for the Treatment of Disease, filed Apr. 10, 2009, the complete contents of which is hereby incorporated by reference, unlocked nucleic acid (UNA), locked nucleic acid (LNA), peptide nucleic acid (PNA), serinol nucleic acid (SNA), twisted intercalating nucleic acid (TINA), anhydrohexitol nucleic acid (HNA), cyclohexenyl nucleic acid (CeNA), D-altritol nucleic acid (ANA) and morpholino nucleic acid (MNA) have also been investigated in splice modulation. Recently, nucleobase-modified AOs containing 2-thioribothymidine, and 5-(phenyltriazol)-2-deoxyuridine nucleotides have been reported to induce exon skipping (Chen S, Le B T, Chakravarthy M, Kosbar T R, Veedu R N. Systematic evaluation of 2′-Fluoro modified chimeric antisense oligonucleotide-mediated exon skipping in vitro. Sci Rep. 2019 Apr. 15; 9(1):6078.). In some embodiments, the antisense oligonucleotides of the invention may be 2′-O-Me RNA/ENA chimera oligonucleotides (Takagi M, Yagi M, Ishibashi K, Takeshima Y, Surono A, Matsuo M, Koizumi M. Design of 2′-O-Me RNA/ENA chimera oligonucleotides to induce exon skipping in dystrophin pre-mRNA. Nucleic Acids Symp Ser (Oxf). 2004; (48):297-8). Other forms of SSOs that may be used to this effect are SSO sequences coupled to small nuclear RNA molecules such as U1 or U7 in combination with a viral transfer method based on, but not limited to, lentivirus or adeno-associated virus (Denti, M A, et al, 2008; Goyenvalle, A, et al, 2004). In some embodiments, the antisense oligonucleotides of the invention are 2′-O-methyl-phosphorothioate nucleotides.

The SSOs of the invention can be synthesized de novo using any of a number of procedures well known in the art. For example, the b-cyanoethyl phosphoramidite method (Beaucage et al., 1981); nucleoside H-phosphonate method (Garegg et al., 1986; Froehler et al., 1986, Garegg et al., 1986, Gaffney et al., 1988). These chemistries can be performed by a variety of automated nucleic acid synthesizers available in the market. These nucleic acids may be referred to as synthetic nucleic acids. Alternatively, SSO's can be produced on a large scale in plasmids (see Sambrook, et al., 1989). SSO's can be prepared from existing nucleic acid sequences using known techniques, such as those employing restriction enzymes, exonucleases or endonucleases. SSO's prepared in this manner may be referred to as isolated nucleic acids.

In some embodiments, the splice switching antisense oligonucleotide of the present invention may be delivered in vivo alone or in association with a vector. In its broadest sense, a “vector” is any vehicle capable of facilitating the transfer of the splice switching antisense oligonucleotide of the present invention to the cells. Preferably, the vector transports the nucleic acid to cells with reduced degradation relative to the extent of degradation that would result in the absence of the vector. In general, the vectors useful in the invention include, but are not limited to, naked plasmids, non-viral delivery systems (electroporation, sonoporation, cationic transfection agents, liposomes, nanoparticules, peptide-bound SSO, etc . . . ), phagemids, viruses, other vehicles derived from viral or bacterial sources that have been manipulated by the insertion or incorporation of the antisense oligonucleotide nucleic acid sequences. Viral vectors are a preferred type of vector and include, but are not limited to nucleic acid sequences from the following viruses: RNA viruses such as a retrovirus (as for example moloney murine leukemia virus and lentiviral derived vectors), harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus. One can readily employ other vectors not named but known to the art. Typically, viral vectors according to the invention include adenoviruses and adeno-associated (AAV) viruses, which are DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAV are derived from the dependent parvovirus AAV (Choi, VW J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hematopoietic cells; and lack of superinfection inhibition thus allowing multiple series of transductions. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by, intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation. In some embodiments, the antisense oligonucleotide nucleic acid sequence is under the control of a heterologous regulatory region, e.g., a heterologous promoter. The promoter can also be, e.g., a viral promoter, such as CMV promoter or any synthetic promoters.

In some embodiments, the splice switching antisense oligonucleotide of the present invention is conjugated to an antibody.

In some embodiments, the antibody suitable is a humanized antibody or a chimeric antibody.

In some embodiments, the antibody has binding affinity for a myeloma-antigen. In some embodiments, the antibody is selected from the group consisting of anti-CD38 antibodies, anti-BCMA antibodies, anti-GPRC5D antibodies and anti-SLAMF7 antibodies.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404,097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein the term “bind” indicates that the antibody has affinity for the surface molecule. The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used hereon, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992. Techniques for conjugating molecule to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, JR., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

In particular embodiments, the antibody is an antibody having binding affinity for CD38.

As used herein, the term “CD38” has its general meaning in the art and refers to the ADP-ribosyl cyclase/cyclic ADP-ribose hydrolase 1.

Thus, in some embodiments, the splice switching antisense oligonucleotide of the present invention is conjugated to an anti-CD38 antibody.

In some embodiments, the antibody is an antibody directed against a least one extracellular domain of CD38.

In some embodiments, the anti-CD38 antibody is selected from the group consisting of isatuximab, daratumumab, MOR202, TAK-079 and felzartamab.

For instance, daratumumab has the heavy chain as set forth in SEQ ID NO:4 and the light chain as set forth in SEQ ID NO:5.

>Daratumumab heavy chain
SEQ ID NO: 4
EVQLLESGGGLVQPGGSLRLSCAVSGFTFNSFAMSWVRQAPGKGLE
WVSAISGSGGGTYYADSVKGRFTISRDNSKNTLYLQMNSLRAEDTA
VYFCAKDKILWFGEPVFDYWGQGTLVTVSSASTKGPSVFPLAPSSK
STSGGTAALGCLVKDYFPEPVTVSWNSGALTSGVHTFPAVLQSSGL
YSLSSVVTVPSSSLGTQTYICNVNHKPSNTKVDKRVEPKSCDKTHT
CPPCPAPELLGGPSVFLFPPKPKDTLMISRTPEVTCVVVDVSHEDP
EVKFNWYVDGVEVHNAKTKPREEQYNSTYRVVSVLTVLHQDWLNGK
EYKCKVSNKALPAPIEKTISKAKGQPREPQVYTLPPSREEMTKNQV
SLTCLVKGFYPSDIAVEWESNGQPENNYKTTPPVLDSDGSFFLYSK
LTVDKSRWQQGNVFSCSVMHEALHNHYTQKSLSLSPGK
>Daratumumab light chain
SEQ ID NO: 5
EIVLTQSPATLSLSPGERATLSCRASQSVSSYLAWYQQKPGQAPRL
LIYDASNRATGIPARFSGSGSGTDFTLTISSLEPEDFAVYYCQQRS
NWPPTFGQGTKVEIKRTVAAPSVFIFPPSDEQLKSGTASVVCLLNN
FYPREAKVQWKVDNALQSGNSQESVTEQDSKDSTYSLSSTLTLSKA
DYEKHKVYACEVTHQGLSSPVTKSFNRGEC

In some embodiments, the antibody is an antibody having binding affinity for BCMA.

As used herein, the term “BCMA” has its general meaning in the art and refers to B Cell Maturation Antigen. Thus, in some embodiments, the splice switching antisense oligonucleotide of the present invention is conjugated to an anti-BCMA antibody.

In some embodiments, the anti-BCMA antibody is selected from the group consisting of belantamab, AMG420, PF-3135, CC-93269, Teclistamad.

In some embodiments, the antibody is an anti-GPRC5D antibody.

As used herein, the term “GPRC5D” has its general meaning in the art and refers to G-protein coupled receptor family C group 5 member D. Thus, in some embodiments, the splice switching antisense oligonucleotide of the present invention is conjugated to an anti-GPRC5D antibody.

In some embodiments, the anti-GPRC5D antibody is Talquetamab.

In some embodiments, the antibody is an anti-SLAMF7 antibody.

As used herein, the term “SLAM-F7”, also known as CD319, has its general meaning in the art and refers to a robust marker of normal plasma cells and malignant plasma cells in multiple myeloma.

In some embodiments, the anti-SLAMF7 antibody is Elotuzumab. Thus, in some embodiments, the splice switching antisense oligonucleotide of the present invention is conjugated to Elotuzumab.

In some embodiments, the method of the present invention is particularly suitable for the treatment of cancer.

In some embodiments, the method of the present invention is particularly suitable for the treatment of liver cancer.

The method of the present invention is particularly suitable for the treatment of B cell malignancies. B-cell malignancies include, but are not limited to, non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL) and related lymphomas (e.g. Waldenstrom's macroglobulinaemia (also called lymphoplasmacytic lymphoma or immunocytoma) or central nervous system lymphomas), leukemias (e.g. acute lymphoblastic leukemia (ALL), chronic lymphocytic leukemia (CLL; also termed B cell chronic lymphocytic leukemia BCLL), hairy cell leukemia and chronic myoblastic leukemia) and myelomas (e.g. multiple myeloma). Additional B cell malignancies include, lymphoplasmacytic lymphoma, plasma cell myeloma, solitary plasmacytoma of bone, extraosseous plasmacytoma, extra-nodal marginal zone B cell lymphoma of mucosa-associated (MALT) lymphoid tissue, nodal marginal zone B cell lymphoma, follicular lymphoma, mantle cell lymphoma, mediastinal (thymic) large B cell lymphoma, intravascular large B cell lymphoma, primary effusion lymphoma, Burkitt's lymphoma/leukemia, grey zone lymphoma, B cell proliferations of uncertain malignant potential, lymphomatoid granulomatosis, and post-transplant lymphoproliferative disorder.

Thus, in some embodiments, the method of the present invention is particularly suitable for the treatment of multiple myeloma, plasmacytoma, plasma cell dyscrasias, plasma cell disorders, Waldenstrom's macroglobulinemia, amyloidosis, B cell lymphoma, diffuse large B cell lymphoma, or plasmablastic lymphoma.

More particularly, the method of the present invention is suitable for the treatment of multiple myeloma.

In some embodiments, the splice switching antisense oligonucleotide of the present invention is administered to the subject in combination with a bh3 mimetic drug.

As used herein, the term “combination” is intended to refer to all forms of administration that provide a first drug together with a further (second, third . . . ) drug. The drugs may be administered simultaneous, separate or sequential and in any order. Drugs administered in combination have biological activity in the subject to which the drugs are delivered. Within the context of the invention, a combination thus comprises at least two different drugs, and wherein one drug is the splice switching antisense oligonucleotide of the present invention and wherein the other drug is the bh3 mimetic drug. In some instance, the combination of the present invention results in the synthetic lethality of the cancer cells.

As used herein, the term “bh3 mimetic drug” has its general meaning in the art and refers to a small compound that antagonizes anti-apoptotic BCL-2 family proteins, resulting in apoptosis induction in cancer cells. The pro-apoptotic BH3 domain consists of an amphipathic α-helix and binds to the hydrophobic groove, which contains BH1, -3 and -4, of anti-apoptotic multidomain proteins, resulting in the release of sequestered pro-apoptotic proteins BAX, BAK, and the activator type BH3-only proteins. Released BAX and BAK activate themselves and/or are activated by released BH3-only proteins to induce apoptosis, suggesting that BH3 peptides or small compounds structurally similar to the BH3 domain could be utilized as therapeutic agents against cancer. In this context, a number of natural or synthetic small molecule inhibitors of anti-apoptotic BCL-2 family proteins were determined, but initially these compounds did not bind to the anti-apoptotic proteins with a high enough affinity and/or activated BAX and BAK to kill target cells efficiently. Among these compounds, ABT-737 mimics the BH3-only proteins by binding to BCL-2, BCL-XL and BCL-W, but not MCL-1, and effectively induces mitochondrion-mediated apoptosis in several cancer cells, particularly MCL-1-suppressed cells. Thus, in some embodiments, the bh3 mimetic drug is selected from the group consisting of ABT-737, ABT-263 (Navitoclax), ABT-199, WEHI-539, BXI-61, BXI-72, GX15-070 (Obatoclax), JY-1-106, and B197C1 (sabutoclax).

In some embodiment, the bh3 mimetic drug is a MCL1 inhibitor.

Thus, in particular embodiments, the splice switching antisense oligonucleotide of the present invention is administered to the subject in combination with an inhibitor of MCL1.

As used herein, the term “MCL1 inhibitor” has its general meaning in the art and refers to a compound that antagonizes anti-apoptotic family member myeloid cell leukemia-1 (MCL-1) resulting in apoptosis induction in cancer cells. MCL-1 was found to have close sequence similarities with BCL-2 and both genes shared “surprising” oncogenic properties: they sustained cell survival but did not promote cell proliferation. Example of MCL1 inhibitors are well known in the art and include but are not limited to compounds disclosed in Bolomsky et al., J Hemato Oncol (2020) 13:173.

In some embodiments, the MCL1 inhibitor is selected from the group consisting of S63845, AMG176, AZD5991, VU661013, Compound 42, b-carboline copper (II) complexes, S7126 (Maritoclax), S8758 (VU661013), S7790 (A-1210477), S7531 (UMI-77), S64315, MIK665, ABBV-467, PRT1419, AMG397,

In particular embodiments, the MCL1 inhibitor is AZD5991.

It will be understood that the total daily usage of the compounds of the present invention (i.e., the SSO of the present invention) will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the splice switching antisense oligonucleotide of the present invention is administered in the form of a pharmaceutical composition. Pharmaceutical compositions of the present invention may also include a pharmaceutically or physiologically acceptable carrier such as saline, sodium phosphate, etc. The compositions will generally be in the form of a liquid, although this need not always be the case. Suitable carriers, excipients and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, cellulose, water syrup, methyl cellulose, methyl and propylhydroxybenzoates, mineral oil, etc. The formulations can also include lubricating agents, wetting agents, emulsifying agents, preservatives, buffering agents, etc. Those of skill in the art will also recognize that nucleic acids are often delivered in conjunction with lipids (e.g., cationic lipids or neutral lipids, or mixtures of these), frequently in the form of liposomes or other suitable micro- or nano-structured material (e.g. micelles, lipocomplexes, dendrimers, emulsions, cubic phases, nanoparticules, etc.).

A further object of the present invention relates to a splice switching antisense oligonucleotide as described above. In some embodiments, the splice switching antisense oligonucleotide comprises the sequence as set forth in SEQ ID NO:3.

In some embodiments, the splice switching antisense oligonucleotide of the invention is conjugated to an antibody.

In some embodiment, the antibody has binding affinity for a myeloma-antigen.

In some embodiments, the antibody is selected from the group consisting of anti-CD38 antibodies, anti-BCMA antibodies, anti-GPRC5D antibodies and anti-SLAMF7 antibodies.

The invention will be further illustrated by the following FIGURES and examples. However, these examples and FIGURES should not be interpreted in any way as limiting the scope of the present invention.

### FIGURES

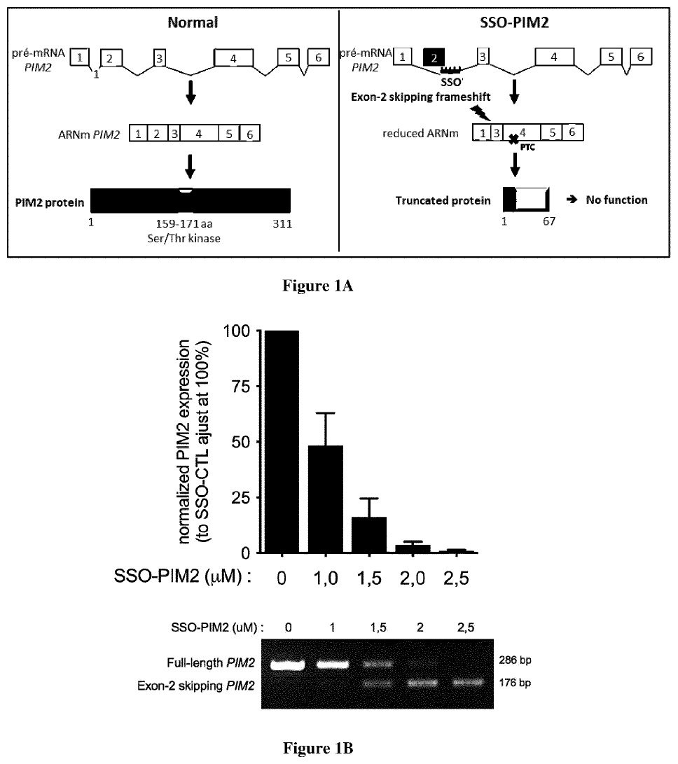

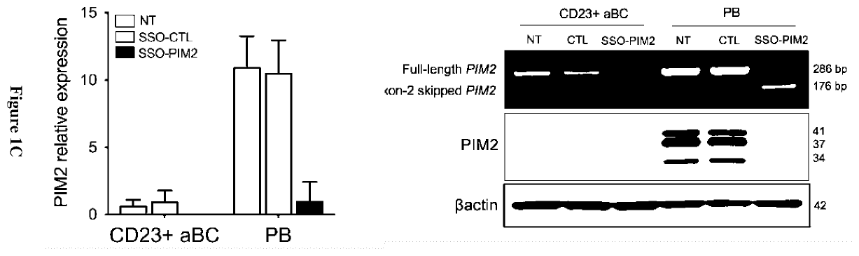

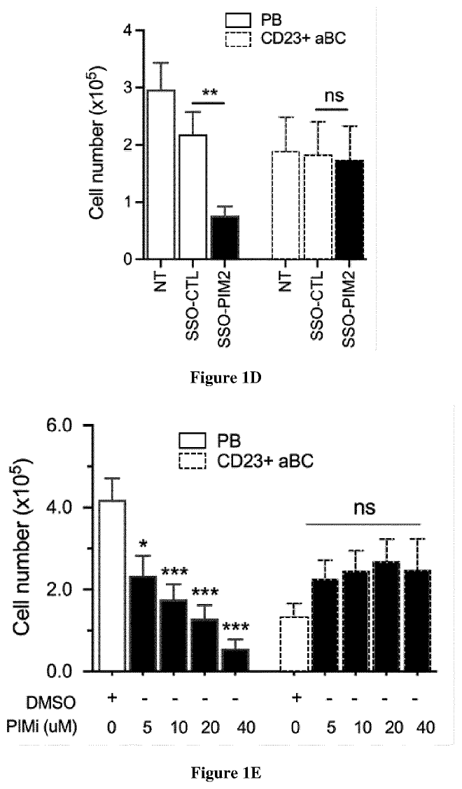

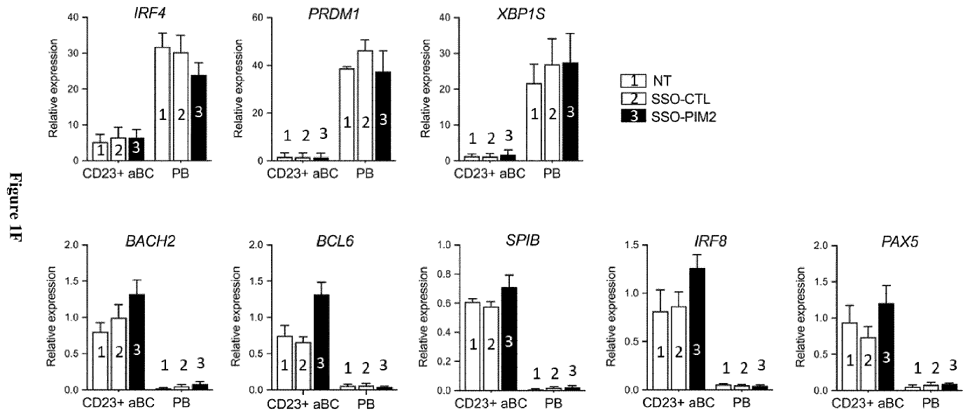

FIG. 1. Specific PIM2 inhibition affects the generation of plasmablasts
A. Diagram explaining SSO-PIM2 action. It binds to the second exon of the gene PIM2 leading in a skip of this exon during transcription and finally results to a frame shift and created an early stop-codon during translation. The protein is so a non-functional truncated protein.
B. Activated B cells were treated at D4 with an increasing dose of SSO-PIM2 and SSO-CTL (control condition) and recovered 48 hours later (at D6). PIM2 mRNA residual expression compared to the control condition adjust at 100% (mean±SD, n=3) and agarose gel of PIM2 PCR, showing exon skipping.
C. At D4, activated B cells were treated with SSO-PIM2 and SSO-CTL (control condition) at 2 μM. CD23+ aBC and PB were sorted at D6. PIM2 mRNA relative expression (mean±SD, n=4) (left) and PIM2 protein expression by western blot (middle) and immunofluorescence (right) after PIM2 inhibition.
D-E. Decrease of PB generation after PIM2 inhibition. Cells were treated with SSO-PIM2 at 2 μM (D) or increase dose of the PIM inhibitor AZD1208 (PIMi as indicated, in μM) (E) and their control condition (SSO-CTL and DMSO respectively). The untreated condition is referred as “NT” (=no treatment). Left: Absolute number of PB obtained at D6 after PIM2 inhibition. Data are representative with bars at mean±SD, n=6. Right: Proportion of PB evaluated at D6 by flow cytometry after treatment with SSO-PIM2 at 2 μM. Data are representative of six independent experiments.
F. Transcription factors (IRF4, PRDM1, XBPIS, BACH2, BCL6, SPIB, IRF8 and PAX5) mRNA relative expression after specific PIM2 inhibition. Data are representative with bars at mean±SD, n=4.

### EXAMPLE

### Stat3-Driven Recruitment of PIM2 During the Plasmablastic Commitment Once BACH2 Release its Inhibition Effect.

To identify genes whose transcription was acutely regulated during the final commitment step we developed our previously described in vitro differentiation model system on human naive B cells (NBCs) giving at day-6 (D6) four cell populations that were explored transcriptionally (data not shown) (Caron et al., 2015; Le Gallou et al., 2012; Pignarre et al., 2021). RNA-seq revealed a striking increase of PIM2 gene expression—but neither for PIM1 nor for PIM3—in PBs (P1 population), and in prePBs as well (P2/CD23− population) compared to B cells diverted from the differentiation (P2/CD23+ and P3 populations); these data were confirmed by qPCR (data not shown). Single-cell RT-qPCRs based on a selection of 90 genes differentially expressed between P2/CD23− and P2/CD23+ found a homogenous expression of PIM2 in prePBs which expression was ranked at the first position before PRDM1 (data not shown). As expected, the western blot showed a correlation between gene expressions and protein levels with a strong expression in prePBs and PBs (data not shown). Interestingly, D4 activated B cells and in particular highly proliferative subset (VPDlo cells), express PIM2 but less than in PB-committed cells (data not shown). We then purified tonsillar-derived cell populations and confirmed that PBs expressed high levels of PIM2—but not PIM1—while naïve, memory and GC B cells did not (data not shown). Investigations of in silico available RNA-seq datasets from mice found similar results (data not shown). Overall, our data revelated that the generation of PBs after B cell activation and differentiation is associated with a sharp elevation of PIM2 kinase.

To determine whether PIM2 gene expression belongs to changes in recruited enhancers and chromatin accessibility described during PC differentiation, we analyzed previous published datasets and ENCODE annotations (Caron et al., 2015; Pignarre et al., 2021). Assay for transposase-accessible chromatin using sequencing (ATAC-seq) showed the dynamic of promoter opening (data not shown). Interestingly, open chromatin regions found in the proximity of PIM2 overlapped with enhancers linked to promoters in the FANTOM5 database (Andersson et al., 2014) but also with regions recently identified in the mouse hematopoietic system by Yoshida et al. who reported that PIM2 was regulated by enhancers and not by its promoter meaning that physical connection exist involving transcription factors that generate functional DNA loops (Yoshida et al., 2019) (data not shown). Thus, enriched chromatin open regions in prePBs cells and PBs revealed a downstream and an upstream region, the latter being a known super-enhancer described in PBs and tumor PCs (data not shown). The comparative analysis of naïve B cells and PBs for genome-wide 5hmC marks showed that read densities increased in both identified ATAC-seq regions, in addition to PIM2 gene body, signing their enhancer function and their activation in PBs (data not shown). High PIM2 expression was found in human PCs signatures from GSEA/MSigDB (GSE13411, for example) and in mouse PCs compared to naïve B cells after in vivo immunization (Minnich et al., 2016; Shi et al., 2015). Both, unsupervised clustering and principal component analysis of our RNA-seq datasets showed that PIM2 was in the top-150 genes characterizing PBs compared to noncommitted B cells (data not shown) (Pignarre et al., 2021). In addition, BACH2 and BCL6 binding sites were found in PIM2 promoter and in the above-described putative regulatory regions, suggesting their negative regulation by B cell identity transcription factors, similar to other PC-identity genes such as IRF4 and PRDM1 (Pignarre et al., 2021) (data not shown). Taken globally, these results demonstrated that PIM2 gene presents an identical expression pattern that PC-identity genes, which pattern is sustained by a specific DNA demethylation activity linked to the PB commitment that starts in prePBs (data not shown) (Caron et al., 2015).

Our ATAC-seq results and previously published data in mice (Yoshida et al., 2019), showed that promoter opening dynamics do not match with the gene expression pattern, i.e. DNA accessibility precedes PIM2 expression, suggesting the presence of a transcriptional regulation within the PIM2 locus (data not shown). By ChIP-seq we identified in human activated NBCs a BACH2 binding site in the PIM2 promoter (Hipp et al., 2017). Three independent experiments using siRNA inhibition, confirmed that BACH2 acts as a PIM2 repressor since downregulation of BACH2 at D4 led to an increased expression of PIM2 at the gene and the protein level (data not shown). In mouse B cells, a variety of growth factor-dependent transcriptional responses targeting PIM2 have been identified (Fox, 2003). In humans, the PB commitment is associated with the downregulation of expression of STATS and STAT6 genes but not STAT3, which remains stable in expression whatever the fate of the cells (data not shown) (Pignarre et al., 2021). Stimulation on generated PBs by various cytokines showed a sharp induction of PIM2 expression by IL-10 and IL-21 and less efficiently by IL-6 (data not shown). All three cytokines recruit the STAT3 pathway whose chemical inhibition induced a dose dependent repression of PIM2 expression in PBs (data not shown). In contrast, IL-2 and interferon α were weak inducers (data not shown)

Taken as a whole, we found that PB differentiation is associated to a sharp PIM2 expression which requires the release of BACH2 repression and the activation of STAT3 signaling (data not shown).

### PIM2 is Required for the Plasmablastic Output of the Differentiation

Plasmablasts emerge during a S phase of the cell cycle after cells were deprived of CD40L, BCR cross-linking and CpG, in presence of IL-2, IL-4 and IL-10 (data not shown) (Caron et al., 2015; Pignarre et al., 2021). To determine the functional involvement of PIM2 in this committal step we had to slightly modify our in vitro differentiation model in order to obtain many cells at D6 with CD23+ post-activated B cells (hereafter referred as CD23⁺ aBC) and PBs, two populations characterized by very distinct PIM2 expression, and devoid of PIM1 (data not shown). In the subsequent experiments we compared systematically the normal setting of the culture model with conditions where PIM2 was inhibited at D4 either by using the chemical pan-PIM inhibitor AZD1208 (hereafter referred as PIMi) or by blocking specifically the expression of the gene PIM2. For this last condition, difficult to handle on primary B cells and PBs we developed an antisense RNA strategy based on a morpholino splice-switching oligonucleotide (SSO) from Gene Tools, LLC (Philomath, OR, US). The SSO-PIM2 after being directly added to the cell culture medium enters the cells and then the nucleus before binding the second exon of PIM2. During the transcription, this binding leads to the jump of the second exon and introduces a stop codon to finally generate a non-functional truncated protein (FIG. 1A). D6 collected PBs under SSO-PIM2 showed a dose-dependent inhibition of PIM2 expression with, as expected, a shift of the size of the amplified gene by PCR (FIG. 1B). We selected 2 μM for our subsequent experiments which produced a near-knockout condition for PIM2 that could last up to 96 h and without affecting PIM1 expression (FIG. 1C). At D6, under the SSO-PIM2 condition, we observed a severe decrease in generated PB (FIG. 1D) and similar results were obtained with PIMi whose effect was dose dependent (FIG. 1E). However, in presence of SSO-PIM2 generated PBs were not affected in terms of expression of key transcription factors involved in B cell differentiation (FIG. 1F). Overall, our data revealed that upregulation of PIM2 in prePBs is necessary for B cells metamorphosis to PBs to take place.

### PIM2 Hinders the Execution of Caspase-3-Driven Apoptosis

Mitochondrial-driven cell death is activated once the mitochondria lose the ability to maintain the inner membrane potential, resulting in the release of proapoptotic proteins into the cytosol. Commitment towards PBs is sustained by an extensive reconfiguration of the epigenetic landscape and of gene expression patterns, both showing that the most prominent function involves cell death and survival (Caron et al., 2015). In accordance with data obtained in vivo in mice for GC B cells (Mayer et al., 2017), the B cell differentiation examined through our original in vitro model (data not shown) revealed that the vast majority of activated B cells diverted from the PB commitment were dead at D6. These cells were rescued from apoptosis with the pan-caspase inhibitor QVD-OPH (data not shown). In contrast, expression levels of the gene CASP3, which encodes the proapoptotic Caspase 3, increased significantly in prePBs and PBs compared to noncommitted B cells (data not shown). We then investigated the different cell subsets generated through the differentiation model at the protein level for factors involved in apoptosis triggered by mitochondria (data not shown). The PBs compared to all other subsets increased the expression of pBAD and XIAP, both proteins counteract the apoptotic pathway and were described in PIM2-induced survival effect (data not shown) (Fox, 2003; Macdonald et al., 2006; Yan et al., 2003). In accordance with this finding and despite a strong increase in procaspase 3 protein (32 kDa size), PBs decrease the autocatalytic activity of Caspase 3 and therefore the production of p19/p17 apoptosis executors as highlighted by the low levels of cleaved PARP (data not shown) (Ponder and Boise, 2019). This effect is caused by the cytoplasmic sequestration of BAD by 14-3-3 protein preventing its mitochondrial localization, which leads to inhibit mitochondrial membrane depolarization maintaining Caspase 9 in an inactivated form and the XIAP protein protected from degradation (data not shown) (Danial, 2008; Datta et al., 2002). The latter plays probably a crucial role in the inhibition of the final execution of apoptosis in PBs since the immunoprecipitation (IP) of XIAP showed the binding of p19 and p17 fragments of Caspase 3, which binding blocks their catalytic activity on procaspase 3 (data not shown) (Riedl et al., 2001; Suzuki et al., 2001). In addition, we found in P2/CD23+ cells a complete degradation of PARP which illustrate the fact that the apoptosis process is consubstantially associated with the B cell terminal differentiation if cells are not otherwise protected (data not shown).

We then inhibited PIM2 which led to an intense activation of Caspase 3 and a decrease of viability of PBs. The latter was rescued by the Caspase 3 inhibitor Q-DEVD-OPH (data not shown). The inhibition of PIM2 induced a strong mitochondrial depolarization (data not shown) leading to the decrease of pBAD and XIAP and the production of cleaved PARP protein as shown for PIMi and confirmed with SSO-PIM2 (data not shown).

Overall, these data show that the kinase PIM2 by its catalytic activity acts in prePBs and PBs on apoptosis by counteracting the Caspase 3 response in cells strongly challenged due to newly acquired secretory capacities.

PIM2 Sustains the G1/S Transition Through Stabilization of Phosphatase CDC25A expression and degradation of cytoplasmic p27^{Kip1}

RNA-seq data comparing prePBs and PBs to noncommitted B cells showed that differentiation is associated with the downregulation of expression of the SMAD3 gene encoding a transcription factor that is a repressor of CDC25A. In line with this result, we found an upregulation of CDC25A expression along with CCND2 and CDK6 and the promotion of cell cycle reentry in prePBs and the emergence of PBs (data not shown). Since PIM2 acts at multiple cellular levels, we wanted to investigate how this kinase impacts this step of the differentiation. By inhibiting PIM2 the number of PBs in S-phase decreased drastically, in a dose-dependent manner for PIMi, suggesting that this kinase may be involved in cell cycle reentry (data not shown). To explore this hypothesis, we investigated the G1/S transition of the cell cycle by using the multiple myeloma-derived cell line XG21 which presents an IL-6/STAT3-dependent PIM2 expression and proliferation (data not shown). To this end, we established a double thymidine block to arrest cells in G1-phase, and then released thymidine before subsequent cell cycle analysis. Unlike the condition without stimulation, the response to IL-6 showed that at 6 h post-release almost all cells reached the S-phase and finally recovered a normal cell cycle at time course 24 h (data not shown). In presence of PIM2 blockade regardless of the type of inhibition, cells remained largely arrested in G1 phase until 16 h before starting to die (data not shown). The transition of G1 to S phase of the cell cycle requires the phosphatase CDC25A. We found a significant increase of CDC25A gene expression in PBs compared to CD23+ aBC and chemical inhibition of the phosphatase led to a dramatic decrease of the number of PBs in S phase (data not shown). We then wondered whether PIM2 has an effect on CDC25A as described for PIM1 which increased the catalytic activity after its binding to the phosphatase (Mochizuki et al., 1999). By western blot we detected CDC25A protein in PBs which disappeared in presence of PIMi (data not shown). PIM2 blockade led also to the downregulation of CDC25A and MKI67 gene expression (data not shown). Taken as a whole, our results showed that PIM2 participates to the stabilization of CDC25A phosphatase in cycling PBs.

Cyclin-dependent kinase inhibitor 1B (p27^{Kip1}) is an enzyme inhibitor that in humans is encoded by the CDKN1B gene and binds to and prevents the activation of cyclin E-CDK2 or cyclin D-CDK4 complexes, and thus controls the cell cycle progression at G1 (Abbastabar et al., 2018; Elledge et al., 1996). The activity of p27^{Kip1} is linked to its nuclear localization. In PBs, the expression of CDKN1B is significantly higher than in CD23+ aBC (data not shown). On the other hand, at the protein level, the expression of p27^{Kip1} was in contrast, lower in PBs than in CD23+ aBC cells, with a clear decrease in the PB nucleus (data not shown). This discrepancy between the gene and the protein expression led us to assess the half-life of p27^{Kip1} with cycloheximide showing a decreased protein stability in PBs due to an enhanced degradation as demonstrated with the proteasome inhibitor MG-132 (data not shown). This finding indicates the onset of an enhanced protein degradation associated with the PB commitment and linked to the occurrence of PIM2 expression. Indeed, the inhibition of PIM2 restored p27^{Kip1} expression in PBs, in particular in the nucleus, and independently to CDKN1B expression (data not shown). In addition, in XG21 cell line treated with cycloheximide we found that p27^{Kip1} half-life increased significantly (data not shown). Altogether, our results demonstrate that PIM2 acts in PBs by degrading cytoplasmic p27^{Kip1} protein sustaining therefore the promotion of the proliferation which is line with a previous description in carcinoma and sarcoma cell lines (Morishita et al., 2008). In prePBs, cell cycle reentry mediates a cycle D2-mediated nuclear export of p27^{Kip1} (Susaki et al., 2007), which is maintained in cycling PBs, and PIM2 allows the degradation of the cytoplasmic protein and its binding to 14-3-3 what prevents the nuclear re-localization (Hnit et al., 2015).

Collectively, the occurrence of PIM2 expression during the PB commitment leads in prePBs to the promotion of the G1/S transition and cell cycle reentry, by stabilizing the expression of CDC25A phosphatase and enhancing cytoplasmic degradation of p27^{Kip1} (data not shown).

PIM2 stabilizes cytoplasmic p21^{Cip1/WAF1} sustaining p21^{Cip1/WAF1}/caspase 3 binding

Like p27^{Kip1}, p21^{Cip1/WAF1} is a tight-binding inhibitor of CDKs and inhibits cell cycle entry into S phase (Elledge et al., 1996). Plasmablast commitment leads to a significant increase of the CDKN1A gene, which encodes p21^{Cip1/WAF1}, and this independently to the expression of TP53 (data not shown). As expected, levels of p21^{Cip1/WAF1} protein were frankly increased in PBs compared to CD23+ aBC but curiously its expression was virtually exclusively cytoplasmic which diverts this factor from its cell cycle repressing function (data not shown). We then wondered whether PIM2 could modify p21^{Cip1/WAF1} expression. Since SSO-PIM2 did not interfere with CDKN1A gene upregulation in PBs (data not shown) we found unlike effect on p27^{Kip1}, that p21^{Cip1/WAF1} protein decreased dramatically its cytoplasmic expression without nuclear relocalization (data not shown). Similar results were obtained in XG21 cell line (data not shown) and taken in account these data we emitted the hypothesis that PIM2 binds p21^{Cip1/WAF1} and participates therefore to the protein stabilization in the cytoplasm. To address this scenario, we developed endogenous protein immunoprecipitation (IP) and found in generated PBs and XG21 cell line as well that PIM2, p21^{Cip1/WAF1} and HSP90β form a cytoplasmic multiprotein complex (data not shown). By using cycloheximide and then proteasome inhibitor MG-132 conditions we demonstrated that p21^{Cip1/WAF1} has a short half-life and is degraded by the proteasome pathway and that PIM2 blockade accelerate the protein degradation (data not shown). Finally, in PBs and XG21 cells treated conjointly by MG-132 and PIMi maintained their p21^{Cip1/WAF1} expression but in a poly-ubiquitinylated form confirming the role of PIM2 in the protein stability by diverting p21^{Cip1/WAF1} from proteasome degradation (data not shown).

The differentiation of immature monocytes is associated with a relocalization of nuclear p21^{Cip1/WAF1} to the cytoplasm where the protein participates to apoptosis blockage (Asada, 1999). In addition, Suzuki et al. ascribed the formation of a Procaspase 3/p21^{Cip1/WAF1} complex in the context of resistance to Fas-mediated cell death owing to the phosphorylation of p21 by protein kinase A (Suzuki et al., 2000). We wanted therefore to explore whether p21^{Cip1/WAF1} may bind Procaspase 3 and developed to this end a FRET approach which detected a signal of energy transfer compatible with such physical interaction. Interestingly, after addition to PIMi in the cell culture we abolished the signal confirming that PIM2 interacts with the p21^{Cip1/WAF1}/Procaspase 3 complex in these cells (data not shown). We then confirmed the binding between p21^{Cip1/WAF1} and Caspase 3 in XG21 cells and in PBs by IP (data not shown). Finally, to confirm the anti-apoptotic role of cytoplasmic p21^{Cip1/WAF1} in PBs we inhibited p21^{Cip1/WAF1} expression by a siRNA targeting CDKN1A in in vitro D4 B cells (data not shown) and found an activation of the execution of apoptosis characterized by the detection of the p17-fragment cleavage of caspase 3 in parallel to the cleavage of PARP (data not shown) and similar results were found in XG21 cells (data not shown). We then used the p21^{Cip1/WAF1} chemical inhibitor UC2288 and observed a dose-dependent increase of Caspase 3 activation in PBs and XG21 (data not shown). This drug, which is an analog of Sorafenib, by inhibiting p21^{Cip1/WAF1} protein expression induced a Caspase 3-mediated apoptosis as confirmed after addition of Caspase 3 inhibitor Q-DEVD-OPH which restores cell viability (data not shown). Taken as a whole, our findings demonstrated that PIM2 upregulation in prePBs is necessary to divert cell from apoptosis in the context of a significant increase of ER stress due to the acquisition of newly secreting functions. The role of PIM2 is to stabilize p21^{Cip1/WAF1} protein in the cytoplasm allowing therefore the formation a multiprotein complex which inhibits Caspase 3 full activation (data not shown).

PIM2 Plays a Role in the Survival of Mature Plasma Cells by Inhibiting Caspase 3 Activation, Notably with the Cytoplasmic Protein p21^{Cip1/WAF1}

Plasma cell maturation is a continuum that spans from PBs to fully mature PCs corresponding to long-lived PCs such as those residing in the bone marrow (BM) PC niche where their survival depends on various extrinsic factors. After demonstrating that PIM2 is essential for the generation of PBs, we wondered whether PIM2 is still expressed in BM PCs. The comparison of PIM2 gene expression in different tonsil-derived B cell populations and PBs, and purified BM PCs obtained from healthy subjects showed that PIM2 increased their expression along the maturation process and other genes explored above modified their expression as expected (data not shown). We then explored PIM2 expression at the protein level in CD138⁺ or BCMA⁺ PCs on BM biopsies from patients free from malignant hemopathies obtained from the Pathology department of our institution (F. Llamas-Gutierrez). Single staining was firstly tested by chromogenic immunohistochemistry (CIH) or immunofluorescence (IF) on paraffin-embedded tissue sections followed by multiple staining by IF. Both, CD138 and BCMA staining allowed to identify PCs with, in some areas, high number of positive cells that possibly were close from CD41-positive megakaryocytes and enriched for IL-6 expression (data not shown). In addition, all observed BM PCs expressed PIM2 with a strong intensity (data not shown). To determine the regulation and functional contribution of PIM2 in mature PCs, we produced CD38^{hi}/CD138⁺ PCs by leaving D7 to D10 generated PBs in culture media containing IL-2, IL-10, IL-6 and IFN-alpha (data not shown). Under this condition, the number of CD138⁺ cells increased progressively while the number of cells in S-phase of the cell cycle decreased gradually with, respectively, 60.3±8.1% and 3.5±0.3% at D10 (data not shown). In parallel, cells decreased and increased, respectively, the expression of CDC25A and CDKN1B with for the latter, p27^{Kip1} protein exclusively detectable in the nucleus at D10 as expected for fully differentiated cells arrested in the cell cycle (data not shown). Altogether, these results are in agreement with the characteristics of early PCs referred hereafter as ePCs. In addition, in these cells, compared to PBs, we observed an increase of PIM2 gene expression as well as its protein level (data not shown). We then demonstrated that in ePCs, and XG21 cells as well, PIM2 expression increase after IL-6 stimulation and stayed at a basal level after IL-6 blockade in culture media (data not shown) demonstrating that PIM2 expression in PCs is primarily dependent on IL-6 signaling. Moreover, we showed the same results by substituting IL-6 by mesenchymal stroma cell (MSC) culture supernatant—which contains a large amount of IL-6—(data not shown) and therefore suggesting that PIM2 expression in BM PCs depends on IL-6 produced by cells present in the BM microenvironment.

We then investigated the other factors described above at the protein levels and detected by western blot in ePCs compared to PBs—and besides p27^{Kip1} and PIM2—a significant elevation of Caspase 3 and p21^{Cip1/WAF1} protein expression (data not shown). To determine whether PIM2 kinase may functionally be involved in mature PCs, we treated ePCs with PIMi and showed a significant increase of mitochondrial release components characterized by the increase of cleaved-Caspase 9 fragment and the decrease of XIAP protein both leading to the activation of Caspase 3 with production of the catalytic active p17 fragment (data not shown). In parallel, we detected after PIMi treatment a decrease of p21^{Cip1/WAF1} expression suggesting that the loss of this factor may be involved in the apoptosis execution in accordance with our findings in PBs (data not shown). Indeed, in ePCs, p21^{Cip1/WAF1} protein—whose gene and protein expression increase compared to PBs—was exclusively detected in the cytoplasm which means that this factor does not participate to cell cycle arrest in PCs (data not shown). In addition, multiple staining on BM biopsies revealed that PIM2⁺ PCs were often positive for cytoplasmic p21^{Cip1/WAF1} as well as Caspase 3 (data not shown). Finally, in ePCs like for PBs, we detected by FRET analysis positive energy transfer between p2^{Cip1/WAF1} and Caspase 3 suggesting a bond between these two factors (data not shown).

The Combination of an Anti-BH3 Mimetic Drug with the Inhibition of PIM2 could Represent an Attractive Therapeutic Option in Multiple Myeloma

Although the involvement of PIM2 in MM has been described for over a decade and has been of interest of many pharma companies, targeting this kinase with chemical inhibitors has proven insufficient. Our work highlights the crucial role of PIM2 for plasma cells and underlines its importance for maintaining their lifespan. In this context, we provide new arguments that suggest the interest of revisiting the targeting of this kinase in MM.

Using data of the Dependency Map (DepMap) portal (https://depmap.org/portal/), we evaluated mRNA expression of PIM2 in silico across >700 different malignant cell lines, including 20 MM cell lines. PIM2 mRNA was highly express in MM cell lines (n=20) and no other tumor types exhibited substantial expression (data not shown). This was specific to PIM2, as PIM1 expression is not overexpress in MM cell lines (data not shown). We confirm the high expression of PIM2 protein in eight MM lines, which correlates well with RNA expression, noting however a large heterogeneity of expression (data not shown) Also, using CRISPR screening of DepMap data, dependency scores of PIM2 underline their specific importance for MM cell survival compared to all cell lines tested, and as compared to PIM1 data not shown). To evaluate potential correlations between PIM2 expression and clinicals outcomes, we analyzed TT2 cohort of the university of Arkansas and within the myeloma patient molecular subgroups. PIM2 expression was significantly higher in three subgroups (MAF, MMSET and Proliferation) compare to the whole MM group which are associated with a poor prognostic (data not shown) (Zhan et al., 2006). We also analyzed the Multiple Myeloma Research Foundation (MMRF) CoMMpass trial, a publicly avaible longitudinal study with accompanying CD138-sorted RNA-seq expression data from 765 patients (https://themmrf.org), and found that PIM2 expression was significantly higher in patients presented translocation (4;14) (t(4;14)), deletion 1q (del1p)—two high-risk cytogenetic abnormalities—and deletion 13 (del13) compared to the whole patients and in contrast, was significantly in patients presented an hyperdiploidy or a translocation t(11;14), two low-risk cytogenetic abnormalities (data not shown) (Bergsagel et al., 2013; Pawlyn and Morgan, 2017; Perrot et al., 2019). Our analysis of the CoMMpass cohort also as PIM2 expression above the cutoff (determined by MaxStat method) in the large dataset correlated with shorter overall survival (P=0.000573) (data not shown). Finally, in the CoMMpass cohort, PIM2 expression was significantly higher at the relapse than at diagnosis, suggesting that PIM2 overexpression could a factor associated with chemoresistance (data not shown). This hypothesis was sustained by analysis of sc-RNAseq data of patients of the KYDAR (KRD-DARA) cohort in which we found that PIM2 is significantly higher in non-responder than responder patients (data not shown) (Cohen et al., 2021). These last result fit well with the fact that PIM2 protein expression increase after proteasome inhibition with drug as carfilzomib as show by western blot in MM cell lines (data not shown), leading to an increase of her prosurvival activity as shown described (Adam et al., 2015). In this context, we showed that low doses of PIM inhibitor potentiated the effects of carfilzomib (data not shown). Taken as whole, these data underline side effects of PIM2 overexpression for patient prognostics and suggest that PIM2 could be a good therapeutic target.

Despite many efforts already undertaken to target this kinase, no chemical inhibitor has yet reached phase 3 due to toxic side effects and insufficient low-dose effects (Raab et al., 2019). In this context, and following our previously described work, we hypothesized a synergistic association between a PIM-inhibitor and an anti-BH3 mimetic drug, which have recently been shown to be effective in MM, and notably inhibitor of MCL1 (Slomp et al., 2019; Wei et al., 2020). MCL1, like PIM2, whose expression depend on IL-6 signaling (Jourdan et al., 2003), is an anti-apoptotic factor which is essential for PC survival (Peperzak et al., 2013) and we found these two factors positively correlated in the TT2 cohort of Arkansas university (data not shown). Unlike PIM2, dependence on MCL1 for cell survival isn't exclusive to tumour PCs but appears to be a universal anti-apoptotic factor (data not shown). By phosphorylating BAD, PIM2 allows BCL2 and BCL-xL to play their role on BAX and/or BAX without acting on MCL1, and therefore we hypothesized that targeting both PIM2 and MCL1 could be an effective strategy to depolarize the mitochondria. To address this scenario, we tested combinaisons of increasing drug-concentration of the PIM inhibitor AZD1208 and the MCL1 inhibitor AZD5991. A hard synergy was obtained by combining these two drugs (data not shown) and revealing a new co-targeting treatment for MM therapy.

### REFERENCES

Throughout this application, various references describe the state of the art to which this invention pertains. The disclosures of these references are hereby incorporated by reference into the present disclosure.

1. A method of reducing expression of PIM2 by exon skipping in a subject in need thereof comprising
administering to the subject an effective amount of at least one splice switching antisense oligonucleotide targeting a splice site of an exon encoding the PIM2, or a splicing regulatory sequence in a pre-mRNA molecule encoding the PIM2, to alter splicing by blocking recognition of said splice site by splicing machinery, thereby inducing the exon skipping.

2. The method of claim 1, wherein the method reduces the expression of PIM2 in B cells.

3. The method of claim 1, wherein the method reduces the expression of PIM2 in malignant cells.

4. The method of claim 1 wherein the at least one splice switching oligonucleotide (SSO) mediates the exon-skipping for a pre-mRNA having at least 3 exons with a targeted internal exon having a number of nucleotides not divisible by 3, thereby inducing a reading frameshift and producing a premature termination codon (PTC) which shortens an open reading frame and/or supports nonsense-mediated mRNA decay (NMD) degradation.

5. The method of claim 1 wherein the at least one splice switching antisense oligonucleotide is an antisense RNA.

6. The method of claim 1 wherein the at least one splice switching antisense oligonucleotide is an antisense DNA.

7. The method of claim 1 wherein the at least one splice switching antisense oligonucleotide targets a PIM2 exon 2 donor splice site.

8. The method of claim 1 wherein the at least one splice switching antisense oligonucleotide is complementary to the nucleic acid sequence as shown in SEQ ID NO:2.

9. The method of claim 1 wherein the at least one splice switching antisense oligonucleotide targets a PIM2 exon 2 donor splice site and comprises the sequence as set forth in SEQ ID NO:3.

10. The method of claim 1 wherein the at least one splice switching antisense oligonucleotide is stabilized.

11. The method of claim 1 wherein the subject suffers from cancer.

12. The method of claim 1 wherein the subject suffers from a liver cancer.

13. The method of claim 1 wherein the subject suffers from a cancer selected from the group consisting of multiple myeloma, plasmacytoma, plasma cell dyscrasias, plasma cell disorders, Waldenström's macroglobulinemia, amyloidosis, B cell lymphoma, diffuse large B cell lymphoma, and plasmablastic lymphoma.

14. The method of claim 1 wherein the subject suffers from multiple myeloma.

15. A splice switching antisense oligonucleotide targeting either a splice site of an exon encoding PIM2, or a splicing regulatory sequence in a pre-mRNA molecule encoding PIM2, wherein the splice switching antisense oligonucleotide alters splicing by blocking recognition of said splice site by splicing machinery thereby inducing exon skipping.

16. The splice switching antisense oligonucleotide according to claim 15 comprising the sequence as set forth in SEQ ID NO:3.

17. The splice switching antisense oligonucleotide according to claim 15, wherein the splice switching antisense oligonucleotide is conjugated to an antibody with binding affinity for a myeloma-antigen.

18. The splice switching antisense oligonucleotide according to claim 17, wherein the antibody is selected from the group consisting of anti-CD38 antibody, anti-BCMA antibody, anti-GPRC5D antibody and anti-SLAMF7 antibody.

19. The method of claim 2, wherein the B cells are plasmablasts and/or plasma cells.

20. The method of claim 3, wherein the malignant cells are malignant B cells and/or malignant plasma cells.
